(19) United States
(12) Patent Application Publication
(10) Pub. No.: US 2012/0128618 A1
(43) Pub. Date: May 24, 2012

(54) COSMETIC CLEANING AGENT HAVING NOVEL MIXTURE OF ACTIVE AGENTS

(75) Inventors: Marcus Claas, Hilden (DE); Bernhard Banowski, Dusseldorf (DE)

(73) Assignee: Henkel AG & Co, KGaA, Dusseldorf (DE)

(21) Appl. No.: 13/359,571

(22) Filed: Jan. 27, 2012

Related U.S. Application Data
(63) Continuation of application No. PCT/EP2010/060963, filed on Jul. 28, 2010.

(30) Foreign Application Priority Data
Jul. 28, 2009 (DE) 10 2009 028 052.9

(51) Int. Cl. A61K 8/73; A61Q 5/12; A61Q 19/10; A61K 8/92; A61Q 5/02

(52) U.S. Cl. 424/70.13; 514/552; 514/549; 510/473

(57) ABSTRACT

A cosmetic cleaning and care agent having good foaming properties includes a) at least one mild anionic surfactant and at least one mild amphoteric/dipolar ionic surfactant, wherein the total quantity of mild anionic and mild amphoteric/dipolar ionic surfactant is 3 to 20% by weight, b) 0.005 to 5% by weight of at least one non-ionic cellulose ether, and c) 0.005 to 5% by weight of at least one non-ionic polymer of the formula (I), where R stands for a hydrogen atom or methyl group, and n stands for an average value of 10 to 1200.

### CROSS-REFERENCES TO RELATED APPLICATIONS

This application is a continuation of PCT/EP2010/060963, filed on Jul. 28, 2010, which claims priority under 35 U.S.C. §119 to DE 10 2009 028 052.9 filed on Jul. 28, 2009, both of which are hereby incorporated by reference.

### FIELD OF THE INVENTION

The present invention generally relates to cosmetic agents and more particularly relates to relates to a cleaning composition based on a special mixture of active substances.

### BACKGROUND OF THE INVENTION

Cosmetic cleaning agents for the skin and hair, such as liquid soaps, shampoos, body washes, bubble baths and shower and washing gels, apart from having good cleaning properties, must also exhibit good compatibility on the skin and mucous membranes and not lead to severe degreasing or skin dryness even with frequent use.

For this reason, attempts have been made for many years to incorporate as many hair- and skin-conditioning agents as possible into cleaning agents.

A large number of these 2-in-1 products are known, yet the storage and stabilizing of the products is extremely difficult and continually presents developers with new challenges. Thus, for example, skin- and hair-conditioning ingredients (oil components or special conditioning polymers) can be incorporated into cosmetic cleaning agents, but their stable dispersion or emulsion over a long period is often problematic.

Another problem can lie in the fact that ionically charged care components interact with oppositely charged ionic components in a negative manner, which in turn can lead to destabilizing of the cleaning compositions.

A further disadvantage of commercial 2-in-1 cleaning compositions lies in the fact that the presence of a large number of care components in the cleaning agent can have a negative effect on its foam properties, which is not generally accepted by consumers.

In EP 1771152 A1, hair-conditioning agents are disclosed which contain high molecular weight ethylene oxides and nonionic cellulose ethers, and which improve the feel of wet hair. The foam properties of the hair-conditioning agents are not disclosed.

The need therefore still exists for cosmetic 2-in-1 skin- and hair-cleaning agents which are stable and have excellent foam properties. Excellent foam properties are understood in particular as a large quantity of foam, rapid formation of the foam and improved sensory and optical properties of the foam.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

The above needs and others are met by a cosmetic cleaning agent, containing at least one mild anionic and at least one mild amphoteric/zwitterionic surfactant in which the total quantity of mild anionic and mild amphoteric/zwitterionic surfactant(s) is 3 to 20 wt. %, 0.005 to 5 wt. % of at least one nonionic cellulose ether, and 0.005 to 5 wt. % of at least one nonionic polymer of formula (I)

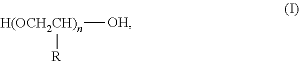

in which R denotes a hydrogen atom or a methyl group and n denotes an average value of 10 to 1200.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present invention provides a cosmetic cleaning agent containing—based on its weight—
a) at least one mild anionic and at least one mild amphoteric/zwitterionic surfactant, wherein the total quantity of mild anionic and mild amphoteric/zwitterionic surfactant(s) is 3 to 20 wt. %,
b) 0.005 to 5 wt. % of at least one nonionic cellulose ether and
c) 0.005 to 5 wt. % of at least one nonionic polymer of formula (I)

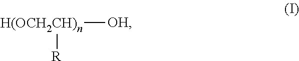

in which R denotes a hydrogen atom or a methyl group and n denotes an average value of 10 to 1200.

Preferred cleaning agents according to the invention contain at least one mild anionic surfactant and at least one mild amphoteric/zwitterionic surfactant in a ratio of anionic surfactant(s) to amphoteric/zwitterionic surfactant(s) of 3:1 to 1:2, preferably 2.5:1 to 1:1.5 and in particular 2:1 to 1:1.

Suitable as anionic surfactants in preparations according to the invention are, in particular,
linear and branched fatty acids with 8 to 30 C atoms (soaps),
ether carboxylic acids of the formula R—O—(CH₂—CH₂O)ₙ—CH₂—COOH, in which R is a linear alkyl group with 8 to 30 C atoms and x=1 to 16,
acyl sarcosides with 8 to 24 C atoms in the acyl group,
acyl taurides with 8 to 24 C atoms in the acyl group,
acyl isethionates with 8 to 24 C atoms in the acyl group,
sulfosuccinic acid mono and dialkyl esters with 8 to 24 C atoms in the alkyl group and sulfosuccinic acid monoalkyl polyoxyethyl esters with 8 to 24 C atoms in the alkyl group and 1 to 6 oxyethyl groups,
linear alkane sulfonates with 8 to 24 C atoms,
linear alpha-olefin sulfonates with 8 to 24 C atoms,
alpha-sulfo fatty acid methyl esters of fatty acids with 8 to 30 C atoms,
alkyl sulfates and alkyl polyglycol ether sulfates of the formula R—O(CH₂—CH₂O)ₓ—OSO₃H, in which R is a preferably linear alkyl group with 8 to 30 C atoms and x=0 or 1 to 12,
mixtures of surface-active hydroxysulfonates,
sulfated hydroxyalkyl polyethylene and/or hydroxyalkylene propylene glycol ethers,
sulfonates of unsaturated fatty acids with 8 to 24 C atoms and 1 to 6 double bonds,
esters of tartaric acid and citric acid with alcohols, which represent addition products of approximately 2-15 molecules of ethylene oxide and/or propylene oxide to fatty alcohols with 8 to 22 C atoms,
alkyl and/or alkenyl ether phosphates of the formula (TI),

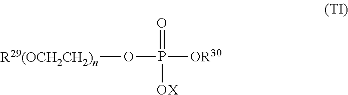

in which R²⁹ preferably denotes an aliphatic hydrocarbon residue with 8 to 30 carbon atoms, R³⁰ denotes hydrogen, a (CH₂CH₂O)ₙR²⁹ residue or X, n denotes numbers from 1 to 10 and X denotes hydrogen, an alkali metal or alkaline earth metal or NR³¹R³²R³³R³⁴, with R³¹ to R³⁴ independently of one another denoting a C₁ to C₄ hydrocarbon residue,
sulfated fatty acid alkylene glycol esters of the formula (TII)

R³⁵CO(AIkO)ₙSO₃M (TII)

in which R³⁵CO denotes a linear or branched, aliphatic, saturated and/or unsaturated acyl residue with 6 to 22 C atoms, Alk denotes CH₂CH₂, CHCH₃CH₂ and/or CH₂CHCH₃, n denotes numbers from 0.5 to 5 and M denotes a cation,
monoglyceride sulfates and monoglyceride ether sulfates of the formula (TIII),

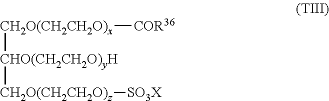

in which R³⁶CO denotes a linear or branched acyl residue with 6 to 22 carbon atoms, x, y and z in total denote 0 or numbers from 1 to 30, preferably 2 to 10, and X denotes an alkali metal or alkaline earth metal. Typical examples of suitable monoglyceride (ether) sulfates within the meaning of the invention are the reaction products of lauric acid monoglyceride, coconut fatty acid monoglyceride, palmitic acid monoglyceride, stearic acid monoglyceride, oleic acid monoglyceride and tallow fatty acid monoglyceride as well as ethylene oxide adducts thereof with sulfur trioxide or chlorosulfonic acid in the form of their sodium salts.
Preferably, monoglyceride sulfates of the formula (TIII) are used, in which R³⁶CO denotes a linear acyl residue with 8 to 18 carbon atoms.

In the cleaning compositions according to the invention, preferably alkyl polyglycol ether sulfates, ether carboxylic acids and/or sulfosuccinic acid monoalkyl polyoxyethyl esters with 8 to 18 C atoms in the alkyl group and 1 to 10, preferably 1 to 4, oxyethyl groups in the molecule are used as mild anionic surfactants.

The anionic surfactant(s) is (are) used in the compositions according to the invention preferably in quantities of 1 to 20 wt. % and in particular in quantities of 2 to 15 wt. %, based on their weight.

Alkyl polyglycol ether sulfates with 10 to 18 C atoms in the alkyl group and 1 to 3 glycol ether groups in the molecule are particularly preferred anionic surfactants owing to their mild properties on the skin.

Those surface-active compounds having at least one quaternary ammonium group and at least one —COO⁽⁻⁾ or —SO₃⁽⁻⁾ group in the molecule are referred to as zwitterionic surfactants. Particularly suitable zwitterionic surfactants are the so-called betaines, such as the N-alkyl-N,N-dimethylammonium glycinates, for example cocoalkyl dimethylammonium glycinate, N-acyl aminopropyl-N,N-dimethylammonium glycinates, for example cocoacyl aminopropyl dimethylammonium glycinate, and 2-alkyl-3-carboxymethyl-3-hydroxyethyl imidazolines with in each case 8 to 18 C atoms in the alkyl or acyl group, and cocoacyl aminoethyl hydroxyethyl carboxymethyl glycinate. Particularly preferred zwitterionic surfactants are the fatty acid amide derivative known by the INCI name Cocamidopropyl Betaine and alkyl betaines having 10 to 20 C atoms in the alkyl group.

Ampholytic surfactants are understood as those surface-active compounds which, as well as a C₈-C₂₄ alkyl or acyl group, contain at least one free amino group and at least one —COOH or —SO₃H group in the molecule and are capable of forming internal salts. Examples of suitable ampholytic surfactants are N-alkyl glycines, N-alkylpropionic acids, N-alkylaminobutyric acids, N-alkyliminodipropionic acids, N-hydroxyethyl-N-alkyl amidopropyl glycines, N-alkyltaurines, N-alkylsarcosines, 2-alkylaminopropionic acids and alkylaminoacetic acids with in each case approximately 8 to 24 C atoms in the alkyl group. Particularly preferred ampholytic surfactants are N-cocoalkyl aminopropionate, cocoacyl aminoethyl aminopropionate and C₁₂-C₁₈ acyl sarcosine.

Particularly preferred amphoteric/zwitterionic surfactants, owing to their mild properties on the skin, are C₈₋₁₈ alkyl betaines, C₈₋₁₈ alkylamido(C₁₋₄alkyl betaines and/or C₈₋₁₈ alkyl amphomonoacetates or amphodiacetates.

The amphoteric/zwitterionic surfactant(s) are used in the compositions according to the invention preferably in quantities of 0.5 to 10 wt. % and in particular in quantities of 1 to 7.5 wt. %, based on their weight.

In a particularly preferred embodiment of the invention, the cosmetic cleaning agents contain a mixture of alkyl ether sulfates and alkylamidoalkyl betaines (as already defined). These surfactants have a particularly mild action on the skin and exhibit advantageous foam formation and foam quality.

To support the gentle cleaning further, it may be preferred if the cleaning agents additionally contain nonionic surfactants. These contain e.g. a polyol group, a polyalkylene glycol ether group or a combination of a polyol and polyglycol ether group as a hydrophilic group. Compounds of this type are, for example,
addition products of 2 to 50 mol ethylene oxide and/or 0 to 5 mol propylene oxide to linear and branched fatty alcohols with 8 to 30 C atoms, to fatty acids with 8 to 30 C atoms and to alkyl phenols with 8 to 15 C atoms in the alkyl group,
addition products of 2 to 50 mol ethylene oxide and/or 0 to 5 mol propylene oxide to linear and branched fatty alcohols with 8 to 30 C atoms, to fatty acids with 8 to 30 C atoms and to alkyl phenols with 8 to 15 C atoms in the alkyl group, end-capped with a methyl or C₂-C₆ alkyl residue, such as e.g. the grades available with the trade names Dehydrol® LS and Dehydrol® LT (Cognis),
addition products of 5 to 60 mol ethylene oxide to castor oil and hydrogenated castor oil,
polyol fatty acid esters, such as e.g. the commercial product Hydagen® HSP (Cognis) or Sovermol grades (Cognis),
alkoxylated triglycerides,
alkoxylated fatty acid alkyl esters of the formula R³⁷CO—(OCH₂CHR³⁸)_{w}OR³⁹, (TIV), in which R³⁷CO denotes a linear or branched, saturated and/or unsaturated acyl residue with 6 to 22 carbon atoms, R³⁸ denotes hydrogen or methyl, R³⁹ denotes linear or branched alkyl residues with 1 to 4 carbon atoms and w denotes numbers from 1 to 20,
amine oxides,
sorbitan fatty acid esters and addition products of ethylene oxide to sorbitan fatty acid esters, such as e.g. the polysorbates,
sugar fatty acid esters and addition products of ethylene oxide to sugar fatty acid esters,
fatty acid N-alkyl glucamides.

Another group of suitable nonionic surfactants are the alkyl polyglucosides. They correspond to formula (I)

R¹O—[G]ₚ

in which R¹ denotes an alkyl and/or alkenyl residue with 4 to 22 carbon atoms, G denotes a sugar residue with 5 or 6 carbon atoms and p denotes numbers from 1 to 10. The index number p in general formula (I) expresses the degree of oligomerization (DP), i.e. the distribution of mono and oligoglycosides, and denotes a number between 1 and 10. Whereas p must always be a whole number in a given compound and here, in particular, can assume the values p=1 to 6, the value p for a particular alkyl oligoglycoside is an analytically determined calculated value, which generally represents a fractional number. Preferably, alkyl and/or alkenyl oligoglycosides with an average degree of oligomerization p of 1.1 to 3.0 are used. From an application point of view, those alkyl and/or alkenyl oligoglycosides having a degree of oligomerization of less than 1.7, and in particular between 1.2 and 1.7, are preferred. The alkyl or alkenyl residue R¹ can be derived from primary alcohols with 4 to 11, preferably 8 to 10, carbon atoms. Most particularly preferred according to the invention are alkyl oligoglucosides based on hydrogenated C_{12/14} coconut alcohol with a DP of 1-3, as are commercially available for example with the INCI name “Coco-Glucoside”.

The nonionic surfactants are used in the compositions according to the invention preferably in quantities of 0.05 to 15 wt. %, more preferably of 0.1 to 10 wt. % and in particular in quantities of 0.5 to 5 wt. %, based on their weight.

As the second essential component b), the cleaning agents according to the invention contain a nonionic cellulose ether, preferably from the group of the ethers and mixed ethers, which is preferably water-soluble.

The term “water-soluble” is understood to refer to those cellulose ethers of which a 1% solution in water at 25° C. is clear or translucent to the human eye.

Preferred cellulose ethers are hydroxyethyl cellulose, hydroxypropyl cellulose and/or hydroxypropyl methyl cellulose and mixtures of these substances. They are commercially available with various viscosities.

The suitable cellulose ethers promote the good foam properties of the cleaning agents according to the invention and provide them with advantageous rheological properties.

It has been found that the cleaning agents according to the invention are particularly stable and high-foaming if they have a higher viscosity.

Preferred cleaning agents therefore have a viscosity in the range of 5000 to 15000 mPas, preferably 6000 to 12000 mPas and in particular 7500 to 10500 mPas (measured in each case with a Haake Viscotester VT550 viscometer; temperature: 20° C., measuring device: cylinder MK-2; shear rate 8/sec.).

Preferred cleaning agents according to the invention contain hydroxypropyl methyl cellulose, as is commercially available for example from Dow Chemicals with the name “Methocel®” or from Hercules with the name “Benecel®” with various degrees of viscosity. A suitable viscosity of the cellulose ethers for the cleaning agents according to the invention is in the range of 100 to 100,000 mPas, and preferably in the range of 2,000 to 10,000 mPas (based on a 2% solution of the particular cellulose ether in water at 20° C.; measured with an Ubbelohde tube viscometer).

Particularly preferred cleaning agents according to the invention contain hydroxypropyl methyl cellulose, as is commercially available for example with the name “Methocel®” (E, F, J, K and 40 series) from Dow Chemical or “Benecel®” from Hercules. The commercial products Methocel® 40-202, Methocel® E4MP, Methocel® 40-100, Methocel® 40-101 and Benecel® MP 330C are especially preferred.

The nonionic cellulose ethers b) are used in the cleaning agents according to the invention preferably in quantities of 0.01 to 4 wt. %, more preferably of 0.05 to 3 wt. % and in particular of 0.1 to 2 wt. %, based on their weight.

Suitable nonionic polymers c) in the cleaning agents according to the invention are preferably also water-soluble, with the term “water-soluble” to be understood as those nonionic polymers c) of which a 1% solution in water at 25° C. is clear or translucent to the human eye.

Suitable nonionic polymers c) correspond to formula (I),

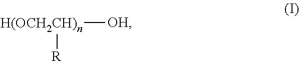

in which R denotes a hydrogen atom or a methyl group and n denotes on average a whole number between 10 and 1200. Particularly preferred polymers c) have a value n of 10 to 900, more preferably of 10 to 600 and in particular of 10 to 400.

In particular, polyethylene glycols are preferred with molecular weights in the range of 300 to 25000 daltons, preferably of 500 to 20000 daltons and in particular of 1000 to 15000 daltons.

It has been found that these special polymers c) support the advantageous rheology of the cleaning agents according to the invention and their foam properties, in particular the fine pores and creaminess of the foam.

It has furthermore been found that the low molecular weight polyethylene glycols described above have an advantageous effect in the cleaning agents according to the invention on the sensory properties of the foam, as the foam leaves no slippery feeling on the skin.

The nonionic polymers c) are used in the cleaning compositions according to the invention preferably in quantities of 0.01 to 4 wt. %, more preferably of 0.05 to 3 wt. % and in particular of 0.1 to 2 wt. %, based on their weight.

In a particularly preferred embodiment of the invention, to support the quantity and density of foam and to improve foam stability further, the cleaning agents according to the invention additionally contain—based on their weight—0.01 to 5 wt. %, preferably 0.05 to 4 wt. %, more preferably 0.1 to 3 wt. % and in particular 0.2 to 2 wt. % of at least one alkanolamide d) of formula (II):

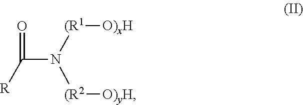

in which R is a C₈-C₂₄ saturated or unsaturated, linear or crosslinked aliphatic group, R¹ and R² are the same or different and form a C₂-C₄ linear or branched aliphatic group, x has a value of 0 to 10 and y has a value of 1 to 10, wherein the sum of x+y is less than or equal to 10.

Specific examples of suitable alkanolamides are, for example, the compounds known by the INCI names “Cocamide MEA”, “Cocamide DEA” and “Cocamide MIPA”.

In another preferred embodiment of the invention, to support the replenishing of lipids and care of the skin, the cleaning agents according to the invention additionally contain monoesters and/or mixtures of monoesters and diesters of glycerol with branched or straight-chained, saturated or unsaturated fatty acids having a C chain length of 8 to 24, preferably of 10 to 18 and in particular of 12 to 16, which have a degree of ethoxylation of 1 to 20, preferably of 2 to 17, particularly preferably of 4 to 13 and in particular of 6 to 10. The ethoxylated glyceryl oleates and glyceryl cocoates are preferred according to the invention, and PEG-7 Glyceryl Cocoate, as is commercially available for example with the name Tegosoft® GC or Cetiol® HE, is particularly preferred.

The pH value of the cleaning compositions according to the invention is ideally in a range that is gentle on the skin, of approximately 4 to 6, in particular in a range of 4.5 to 5.5. Apart from the above-mentioned constituents, the cleaning compositions can contain a series of other optional constituents. Other active substances that have cosmetic care properties are preferably added to the cleaning compositions to support the conditioning of the skin and/or hair during the cleaning process. As such, in particular cosmetically suitable oil components, plant extracts and/or humectants may be mentioned as other preferred optional components.

Suitable oil components can be selected from mineral, natural or synthetic oil components such as petrolatum, paraffins, silicones, fatty alcohols, fatty acids, fatty acid esters and natural oils of plant and animal origin. They are used in the cleaning compositions preferably in a quantity of 0.01 to 10 wt. %, particularly preferably of 0.05 to 5 wt. % and in particular of 0.2 to 3 wt. %, based on their total weight (the quantitative data being based on the total content of all oil components in the cleaning agent according to the invention).

The term silicone oils is understood by the person skilled in the art as a number of structures of organosilicon compounds, which are preferably selected from at least one representative of the organosilicon compounds which is made up of:
(i) polyalkyl siloxanes, polyaryl siloxanes, polyalkyl aryl siloxanes, which are volatile or non-volatile, straight-chained, branched or cyclic, crosslinked or non-crosslinked;
(ii) polysiloxanes, which contain in their general structure one or more organofunctional groups that are selected from:
   a) substituted or unsubstituted aminated groups;
   b) (per)fluorinated groups;
   c) thiol groups;
   d) carboxylate groups;
   e) hydroxylated groups;
   f) alkoxylated groups;
   g) acyl oxyalkyl groups;
   h) amphoteric groups;
   i) bisulfite groups;
   j) hydroxyacyl amino groups;
   k) carboxy groups;
   I) sulfonic acid groups; and
   m) sulfate or thiosulfate groups;
(iii) linear polysiloxane (A)—polyoxyalkylene (B) block copolymers of the type (A-B)ₙ with n>3;
(iv) graft silicone polymers with a non-silicone-containing organic backbone, which consist of an organic main chain made up of organic monomers that do not contain any silicone, on which at least one polysiloxane macromer has been grafted in the chain and optionally on at least one end of the chain;
(v) grafted silicone polymers with a polysiloxane backbone, on which non-silicone-containing organic monomers have been grafted, which have a polysiloxane main chain on which at least one organic macromer that does not contain any silicone has been grafted in the chain and optionally on at least one end thereof, such as e.g. the commercial product Abil B 8832 from Degussa marketed with the INCI name Bis-PEG/PPG-20/20 Dimethicone;
(vi) or mixtures thereof.

Preferred hair-conditioning silicones are selected from dimethicones, amodimethicones or dimethiconols.

As fatty acids it is possible to use linear and/or branched, saturated and/or unsaturated fatty acids with 6-30 carbon atoms. Fatty acids with 10-22 carbon atoms are preferred. These would include, for example, the isostearic acids, such as the commercial products Emersol® 871 and Emersol® 875, and isopalmitic acids, such as the commercial product Edenor® IP 95, and all other fatty acids marketed with the trade names Edenor® (Cognis). Other typical examples of these fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and technical mixtures thereof.

The fatty acid blends that are obtainable from coconut oil or palm oil are usually particularly preferred; as a rule, the use of stearic acid is preferred in particular.

As fatty alcohols it is possible to use saturated, mono- or polyunsaturated, branched or unbranched fatty alcohols with C₆-C₃₀, preferably C₁₀-C₂₂ and most particularly preferably C₁₂-C₂₂ carbon atoms. Within the meaning of the invention it is possible to use, for example, decanol, octanol, octenol, dodecenol, decenol, octadienol, dodecadienol, decadienol, oleyl alcohol, erucyl alcohol, ricinoleyl alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, and the Guerbet alcohols thereof, this list being intended to be of an exemplary and non-limiting nature. However, the fatty alcohols are preferably derived from natural fatty acids, generally starting by obtaining them from the esters of the fatty acids by reduction. It is likewise possible to use those fatty alcohol blends which are produced by reduction of naturally occurring triglycerides, such as beef tallow, palm oil, peanut oil, rape seed oil, cottonseed oil, soybean oil, sunflower oil and linseed oil or fatty acid esters resulting from their transesterification products with corresponding alcohols, and thus represent a mixture of different fatty alcohols. Substances of this type are commercially available, for example, with the names Stenol®, e.g. Stenol® 1618 or Lanette®, e.g. Lanette® 0 or Lorol®, e.g. Lorol® C8, Lorol® C14, Lorol® C18, Lorol® C₈₋₁₈, HD-Ocenol®, Crodacol®, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. It is, of course, also possible to use wool alcohols, as are commercially available, for example, with the names Corona®, WhiteSwan®, Coronet® or Fluilan®.

Solid paraffins or isoparaffins, carnauba waxes, beeswaxes, candelilla waxes, ozokerites, ceresin, cetaceum, sunflower wax, fruit waxes, such as e.g. apple wax or citrus wax, and microwaxes of PE or PP can be used as natural or synthetic waxes. These waxes are available for example via Kahl & Co., Trittau.

The natural and synthetic cosmetic oils include, for example:
Liquid paraffin oils, isoparaffin oils and synthetic hydrocarbons as well as di-n-alkyl ethers with a total of between 12 and 36 C atoms, in particular 12 to 24 C atoms, such as e.g. di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether and n-hexyl n-undecyl ether as well as di-tert-butyl ether, diisopentyl ether, di-3-ethyl decyl ether, tert.-butyl n-octyl ether, isopentyl n-octyl ether and 2-methylpentyl n-octyl ether. The compounds 1,3-di-(2-ethylhexyl)cyclohexane (Cetiol® S) and di-n-octyl ether (Cetiol® OE) available as commercial products may be preferred.
Ester oils. The term “ester oils” is to be understood as the esters of C₆-C₃₀ fatty acids with C₂-C₃₀ fatty alcohols. The monoesters of fatty acids with alcohols having 2 to 24 C atoms are preferred. Examples of fatty acid fractions used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid, and technical mixtures thereof.
Examples of the fatty alcohol fractions in the ester oils are isopropyl alcohol, caproyl alcohol, capryl alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol, as well as mixtures thereof.
Particularly preferred are isopropyl myristate (Rilanit® IPM), isononanoic acid C₁₆₋₁₈ alkyl esters (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprate/caprylate (Cetiol® LC), n-butyl stearate, oleyl erucate (Cetiol® J 600), isopropyl palmitate (Rilanit® IPP), oleyl oleate (Cetiol®, lauric acid hexyl ester (Cetiol® A), di-n-butyl adipate (Cetiol® B), myristyl myristate (Cetiol® MM), cetearyl isononanoate (Cetiol® SN) and oleic acid decyl ester (Cetiol® V).
Dicarboxylic acid esters, such as di-n-butyl adipate, di(2-ethylhexyl) adipate, di(2-ethylhexyl) succinate and diisotridecyl acelaat and diol esters, such as ethylene glycol dioleate, ethylene glycol diisotridecanoate, propylene glycol di(2-ethylhexanoate), propylene glycol diisostearate, propylene glycol dipelargonate, butanediol diisostearate and neopentyl glycol dicaprylate,
Symmetrical, asymmetrical or cyclic esters of carbonic acid with fatty alcohols, glycerol carbonate or dicaprylyl carbonate (Cetiol® CC),
Tri-fatty acid esters of saturated and/or unsaturated, linear and/or branched fatty acids with glycerol,
Optionally ethoxylated fatty acid partial glycerides, which are optionally ethoxylated monoglycerides, diglycerides and technical mixtures thereof. When technical products are used, small quantities of triglycerides may still be contained as a result of their production process. The partial glycerides preferably follow the formula

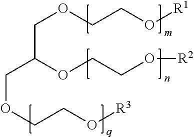

in which R¹, R² and R³, independently of one another, denote hydrogen or a linear or branched, saturated and/or unsaturated acyl residue with 6 to 22, preferably 12 to 18, carbon atoms with the proviso that at least one of these groups denotes an acyl residue and at least one of these groups denotes hydrogen. The sum of (m+n+q) denotes 0 or numbers from 1 to 100, preferably 0 or 5 to 25. Preferably, R1 denotes an acyl residue and R2 and R3 denote hydrogen and the sum of (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid, as well as technical mixtures thereof. Oleic acid monoglycerides are preferably used.

In a particularly preferred embodiment of the invention, the cleaning agents according to the invention can contain a vegetable oil as the oil component.

As natural oils, for example, amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, rosehip seed oil, hazelnut oil, elderberry seed oil, blackcurrant seed oil, jojoba oil, cocoa butter, linseed oil, macadamia nut oil, maize germ oil, almond oil, marula oil, evening primrose oil, olive oil, palm oil, peach kernel oil, rape oil, rice oil, sea buckthorn fruit oil, sea buckthorn seed oil, sesame oil, shea butter, soybean oil, sunflower oil, grape kernel oil, walnut oil or wild rose oil are suitable.

Particularly preferred are avocado oil, apricot kernel oil, rosehip seed oil, jojoba oil, cocoa butter, almond oil, olive oil, peach kernel oil, shea butter, sunflower oil and grape seed oil.

Suitable plant extracts are to be understood as extracts that can be produced from all parts of a plant.

These extracts are generally produced by extraction of the entire plant. In individual cases, however, it may also be preferred to produce the extracts exclusively from flowers and/or leaves of the plant.

Preferred for use in the cleaning agents according to the invention are, above all, the extracts of green tea, white tea, oak bark, stinging nettle, witch hazel, hops, chamomile, burdock root, horsetail, hawthorn, lime blossom, lychee, almond, aloe vera, fir needle, horse chestnut, sandalwood, juniper, coconut, mango, apricot, lime, wheat, kiwi, melon, orange, grapefruit, sage, rosemary, birch, mallow, cuckoo flower, wild thyme, yarrow, thyme, melissa, rest harrow, coltsfoot, marsh mallow, ginseng, ginger root, Echinacea purpurea, Olea europea, Foeniculum vulgaris and Apim graveolens.

Water, alcohols and mixtures thereof can be used as extracting agents to produce the above-mentioned plant extracts. Among the alcohols here, low alcohols such as ethanol and isopropanol, but in particular polyhydric alcohols such as ethylene glycol and propylene glycol, are preferred both as a single extracting agent and in a mixture with water. Plant extracts based on water/propylene glycol in a ratio of 1:10 to 10:1 have proved particularly suitable.

The plant extracts can be used both in pure form and in dilute form. Where they are used in dilute form, they generally contain approx. 2-80 wt. % of active substance and, as solvent, the extracting agent or mixture of extracting agents used to obtain them.

In addition, it may prove advantageous if the compositions according to the invention contain humectants or penetration enhancers and/or swelling agents (M). These auxiliary substances ensure better penetration of active substances into the keratin fibers or help the keratin fibers to swell. The suitable humectants or penetration enhancers and/or swelling agents (M) include, for example, urea and urea derivatives, guanidine and its derivatives, arginine and its derivatives, water glass, imidazole and its derivatives, histidine and its derivatives, benzyl alcohol, glycerol, glycol and glycol ethers, propylene glycol and propylene glycol ethers, for example propylene glycol monoethyl ether, carbonates, hydrogen carbonates, diols and triols, and in particular 1,2-diols and 1,3-diols, such as e.g. 1,2-propanediol, 1,2-pentanediol, 1,2-hexanediol, 1,2-dodecanediol, 1,3-propanediol, 1,6-hexanediol, 1,5-pentanediol and 1,4-butanediol.

Glycerol is particularly suitable.

The humectants or penetration enhancers and/or swelling agents (M) are used in the cleaning compositions according to the invention preferably in quantities of 0.05 to 5 wt. % and in particular in quantities of 0.1 to 3 wt. %, based on the total composition.

As other optional components, cationic polymers can be used in the cleaning compositions according to the invention.

Suitable cationic polymers are polymers that have groups in the main and/or side chain that can be “temporarily” or “permanently” cationic. Those polymers which, regardless of the pH value of the agent, have a cationic group are referred to as “permanently cationic”. These are generally polymers that contain a quaternary nitrogen atom, for example in the form of an ammonium group.

Preferred cationic groups contain quaternary ammonium groups. In particular, those polymers in which the quaternary ammonium group is bonded via a C₁₋₄ hydrocarbon group to a polymer main chain made up of acrylic acid, methacrylic acid or derivatives thereof have proved particularly suitable.

Homopolymers containing a grouping of the general formula (VI),

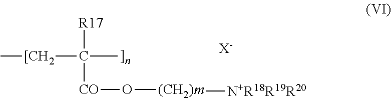

in which R¹⁷═—H or —CH₃, R¹⁸, R¹⁹ and R²⁰, independently of one another, are selected from C₁₋₄ alkyl, alkenyl or hydroxyalkyl groups, m=1, 2, 3 or 4, n is a natural number and X⁻ is a physiologically acceptable organic or inorganic anion, and copolymers consisting substantially of the monomer units shown in formula (VI) and nonionogenic monomer units, are particularly preferred cationic polymers.

Within the framework of these polymers, those for which at least one of the following conditions applies are preferred:

R¹⁷ denotes a methyl group

R¹⁸, R¹⁹ and R²⁰ denote methyl groups

m has the value 2.

Suitable as physiologically acceptable counter-ions X— are, for example, halide ions, sulfate ions, phosphate ions, methosulfate ions and organic ions such as lactate, citrate, tartrate and acetate ions. Halide ions, in particular chloride, are preferred.

Suitable homo- or copolymers that are derived from formula (VI) are, for example, those that are commercially available with the trade names Salcare® SC 95, Salcare® SC 96 and Salcare® SC 92.

Other preferred cationic polymers are, for example,
quaternized cellulose derivatives, as are commercially available with the names Celquat® and Polymer JR®. The compounds Celquat® H 100, Celquat® L 200 and Polymer JR®400 are preferred quaternized cellulose derivatives,
hydrophobically modified cellulose derivatives, for example the cationic polymers marketed with the trade name SoftCat®,
cationic alkyl polyglycosides,
cationized honey, for example the commercial product Honeyquat® 50,
cationic guar derivatives, such as in particular the products marketed with the trade names Cosmedia® Guar and Jaguar®,
polysiloxanes with quaternary groups, such as e.g. the commercially available products Q2-7224 (manufacturer: Dow Corning; a stabilized trimethylsilyl amodimethicone), Dow Corning® 929 Emulsion (containing a hydroxylamino-modified silicone, which is also referred to as amodimethicone), SM-2059 (manufacturer: General Electric), SLM-55067 (manufacturer: Wacker) and Abil® Quat 3270 and 3272 (manufacturer: Th. Goldschmidt; diquaternary polydimethyl siloxanes, Quaternium-80),
polymeric dimethyldiallylammonium salts and copolymers thereof with esters and amides of acrylic acid and methacrylic acid. The products commercially available with the names Merquat®100 (poly(dimethyldiallylammonium chloride)) and Merquat®550 (dimethyldiallylammonium chloride-acrylamide copolymer) are examples of these cationic polymers,
copolymers of vinylpyrrolidone with quaternized derivatives of diallyl aminoalkyl acrylate and methacrylate, such as e.g. vinylpyrrolidone/dimethylaminoethyl methacrylate copolymers quaternized with diethyl sulfate. These compounds are commercially available with the names Gafquat®734 and Gafquat®755,
vinylpyrrolidone/vinylimidazolium methochloride copolymers, as are available with the names Luviquat® FC 370, FC 550, FC 905 and HM 552,
quaternized polyvinyl alcohol,
   and the polymers with quaternary nitrogen atoms in the polymer main chain known by the names
polyquaternium 2,
polyquaternium 17,
polyquaternium 18 and
polyquaternium 27.

Likewise, it is possible to use the polymers known by the names Polyquaternium-24 (commercial product, e.g. Quatrisoft® LM 200) as cationic polymers. The copolymers of vinylpyrrolidone can also be used, as are available as the commercial products Copolymer 845 (manufacturer: ISP), Gaffix® VC 713 (manufacturer: ISP), Gafquat® ASCP 1011, Gafquat® HS 110, Luviquat®8155 and Luviquat® MS 370.

Other cationic polymers are the so-called “temporarily cationic” polymers. These polymers generally contain an amino group, which is present at certain pH values as a quaternary ammonium group and thus in cationic form. For example, chitosan and its derivatives, as are freely commercially available e.g. with the trade names Hydagen® CMF, Hydagen® HCMF, Kytamer® PC and Chitolam® NB/101, are preferred. Chitosans are deacetylated chitins, which are commercially available in different degrees of deacetylation and different degrees of degradation (molecular weights).

The cationic polymer(s) is (are) used in the cleaning compositions according to the invention preferably in quantities of 0.2 to 3 wt. % and in particular of 0.5 to 2 wt. %, based on their total weight.

The cleaning compositions according to the invention are suitable as cosmetic compositions for cleaning the skin and/or hair, such as e.g. hair shampoos, shower gels, body washes, washing gels, facial cleansers, hand-washing agents and/or bubble baths. As such, they can contain additional active substances, auxiliary substances and additives which are described below.

The cleaning agents according to the invention can contain cationic surfactants of the type of the quaternary ammonium compounds, ester quats and amidoamines. Preferred quaternary ammonium compounds are ammonium halides, in particular chlorides and bromides, such as alkyl trimethylammonium chlorides, dialkyl dimethylammonium chlorides and trialkyl methylammonium chlorides, e.g. cetyl trimethylammonium chloride, stearyl trimethylammonium chloride, distearyl dimethylammonium chloride, lauryl dimethylammonium chloride, lauryl dimethyl benzyl ammonium chloride and tricetyl methylammonium chloride, and the imidazolium compounds known by the INCI names Quaternium-27 and Quaternium-83. The long alkyl chains of the above-mentioned surfactants preferably have 10 to 18 carbon atoms.

Ester quats are known substances which contain both at least one ester function and at least one quaternary ammonium group as structural elements. Preferred ester quats are quaternized ester salts of fatty acids with triethanolamine, quaternized ester salts of fatty acids with diethanol alkylamines and quaternized ester salts of fatty acids with 1,2-dihydroxypropyl dialkylamines. Products of this type are marketed, for example, with the trade marks Stepantex®, Dehyquart® and Armocare©. The products Armocare® VGH-70, an N,N-bis(2-palmitoyloxyethyl)dimethylammonium chloride, and Dehyquart® F-75, Dehyquart® C-4046, Dehyquart® L80 and Dehyquart® AU-35 are examples of these ester quats.

The alkylamidoamines are generally produced by amidation of natural or synthetic fatty acids and fatty acid blends with dialkylaminoamines. A particularly suitable compound from this group of substances is stearamidopropyl dimethylamine, which is commercially available with the name Tegoamid® S 18.

The cationic surfactants are preferably used in quantities of 0.05 to 10 wt. %, based on the overall agent. Quantities of 0.1 to 5 wt. % are particularly preferred.

In another preferred embodiment, the action of the compositions according to the invention can be increased by emulsifiers. These emulsifiers are, for example,
addition products of 4 to 30 mol ethylene oxide and/or 0 to 5 mol propylene oxide to linear fatty alcohols with 8 to 22 C atoms, to fatty acids with 12 to 22 C atoms and to alkylphenols with 8 to 15 C atoms in the alkyl group,
ethylene oxide and polyglycerol addition products to methyl glucoside fatty acid esters, fatty acid alkanolamides and fatty acid glucamides,
C₈-C₂₂ alkyl monoglycosides and oligoglycosides and their ethoxylated analogs, wherein degrees of oligomerization of 1.1 to 5, in particular 1.2 to 2.0, and glucose as sugar component are preferred,
mixtures of alkyl (oligo)glucosides and fatty alcohols, for example the commercially available product Montanov® 68,
addition products of 5 to 60 mol ethylene oxide to castor oil and hydrogenated castor oil,
partial esters of polyols having 3-6 carbon atoms with saturated fatty acids having 8 to 22 C atoms,
sterols. As sterols, a group of steroids are understood, which carry a hydroxyl group on the C atom 3 of the steroid backbone and are isolated both from animal tissue (zoosterols) and from plant fats (phytosterols). Examples of zoosterols are cholesterol and lanosterol. Examples of suitable phytosterols are ergosterol, stigmasterol and sitosterol. Sterols are also isolated from fungi and yeasts, the so-called mycosterols.
phospholipids. These are understood above all as the glucose phospholipids, which are obtained e.g. as lecithins or phosphatidylcholines from e.g. egg yolk or plant seeds (e.g. soybeans).
fatty acid esters of sugars and sugar alcohols, such as sorbitol,
polyglycerols and polyglycerol derivatives, such as e.g. polyglycerol poly-12-hydroxystearate (commercial product Dehymuls® PGPH),
linear and branched fatty acids with 8 to 30 C atoms and Na, K, ammonium, Ca, Mg and Zn salts thereof.

The emulsifiers are used preferably in quantities of 0.1-25 wt. %, in particular 0.5-15 wt. %, based on the overall agent.

In principle, nonionogenic emulsifiers with an HLB value of 8 to 18 can be used.

Nonionogenic emulsifiers with an HLB value of 10-15 may be preferred.

In another embodiment of the invention, the compositions according to the invention can additionally contain protein hydrolyzates and/or derivatives thereof to further support their skin- and hair-care action.

Protein hydrolyzates are product mixtures that are obtained by acid-, base- or enzyme-catalyzed degradation of proteins.

In the cleaning agents according to the invention, protein hydrolyzates of both plant and animal origin can be used.

Animal protein hydrolyzates are, for example, elastin, collagen, keratin, silk and milk protein hydrolyzates, which can also be present in the form of salts. These products are marketed, for example, with the trade marks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Protein hydrolyzates of plant origin, e.g. soybean, almond, rice, pea, potato and wheat protein hydrolyzates, are preferred. These products are available, for example, with the trade marks Gluadin® (Cognis), DiaMin® (Diamalt), Lexein® (Inolex) and Crotein® (Croda).

Although the use of protein hydrolyzates as such is preferred, amino acid mixtures or individual amino acids obtained in another way, such as e.g. arginine, lysine, histidine or pyroglutamic acid, may optionally also be used in their place. The use of derivatives of protein hydrolyzates is also possible, for example in the form of their fatty acid condensation products. These products are marketed, for example, with the names Lamepon® (Cognis), Gluadin® (Cognis), Lexein® (Inolex), Crolastin® (Croda) or Crotein® (Croda).

In the cleaning agents according to the invention, cationized protein hydrolyzates can also be contained, wherein the base protein hydrolyzate can come from an animal source, for example from collagen, milk or keratin, a plant source, for example from wheat, maize, rice, potatoes, soybean or almonds, from marine life forms, for example from fish collagen or algae, or from protein hydrolyzates obtained by biotechnology.

The protein hydrolyzates on which the cationic derivatives are based can be obtained from the corresponding proteins by means of chemical, in particular alkaline or acid, hydrolysis, by enzymatic hydrolysis and/or a combination of the two types of hydrolysis. The hydrolysis of proteins generally provides a protein hydrolyzate with a molecular weight distribution of approximately 100 daltons up to several thousand daltons. Those cationic protein hydrolyzates based on a protein fraction having a molecular weight of 100 to 25000 daltons, preferably 250 to 5000 daltons, are preferred

Cationic protein hydrolyzates are also understood to include quaternized amino acids and mixtures thereof. The quaternizing of the protein hydrolyzates or amino acids is often performed using quaternary ammonium salts, such as e.g. N,N-dimethyl-N-(n-alkyl)-N-(2-hydroxy-3-chloro-n-propyl)ammonium halides. The cationic protein hydrolyzates can also be further derivatized. Typical examples of the cationic protein hydrolyzates and derivatives are the commercially available products known by the INCI names: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein and Quaternium-79 Hydrolyzed Wheat Protein.

The plant-based cationic protein hydrolyzates and derivatives are most particularly preferred.

The protein hydrolyzates and derivatives thereof are used preferably in quantities of 0.01-10 wt. %, based on the overall agent. Quantities of 0.1 to 5 wt. %, in particular 0.1 to 3 wt. %, are most particularly preferred.

The combination of the composition according to the invention with vitamins, provitamins and vitamin precursors and derivatives thereof has also proved advantageous.

Vitamins, provitamins and vitamin precursors that are generally assigned to the groups A, B, C, E, F and H are preferred.

The group of substances referred to as vitamin A includes retinol (vitamin A₁) and 3,4-didehydroretinol (vitamin A₂). β-Carotene is the provitamin of retinol. Suitable as vitamin A component according to the invention are, for example, vitamin A acid and esters thereof, vitamin A aldehyde and vitamin A alcohol and esters thereof, such as the palmitate and acetate. The vitamin A component is used preferably in quantities of 0.05-1 wt. %, based on the overall preparation.

The vitamin B group or vitamin B complex includes, inter alia:
vitamin B₁ (thiamin);
vitamin B₂ (riboflavin);
vitamin B₃. This name often includes the compounds nicotinic acid and nicotinamide (niacinamide). Nicotinamide, which is used preferably in quantities of 0.05 to 1 wt. %, based on the overall agent, is preferred.
vitamin B₅ (pantothenic acid and panthenol). In the context of this group, panthenol is preferably used. Suitable derivatives of panthenol are, in particular, the esters and ethers of panthenol as well as cationically derivatized panthenols. Individual representatives are, for example, panthenol triacetate, panthenol monoethyl ether and the monoacetate thereof as well as the cationic panthenol derivatives disclosed in WO 92/13829. The aforementioned compounds of the vitamin B₅ type are used preferably in quantities of 0.05-10 wt. %, based on the overall agent. Quantities of 0.1-5 wt. % are particularly preferred.
vitamin B₆ (pyridoxine as well as pyridoxamine and pyridoxal).
vitamin C (ascorbic acid). The usual quantity of vitamin C used is 0.1 to 3 wt. %, based on the overall agent. Use in the form of the palmitic ester, glucosides or phosphates may be preferred. Use in combination with tocopherols may likewise be preferred.
vitamin E (tocopherols, in particular α-tocopherol). Tocopherol and its derivatives, including in particular the esters, such as the acetate, nicotinate, phosphate and succinate, are used preferably in quantities of 0.01 to 1 wt. %, based on the overall agent.
vitamin F. The term “vitamin F” is usually understood to mean essential fatty acids, in particular linoleic acid, linolenic acid and arachidonic acid.
vitamin H. The compound (3aS,4S,6aR)-2-oxohexahydrothienol[3,4-d]-imidazole-4-valeric acid is referred to as vitamin H, but its trivial name biotin has now become accepted. Biotin is used preferably in quantities of 0.0001 to 1.0 wt. %, in particular in quantities of 0.001 to 0.01 wt. %.

The use of vitamins, provitamins and vitamin precursors from groups A, B, E and H is preferred. Panthenol and derivatives thereof and nicotinamide and biotin are particularly preferred.

Furthermore, in a preferred embodiment of the invention, a UV filter (I) can additionally be used. The UV filters to be used are not subject to any general restrictions in terms of their structure and their physical properties. Rather, all UV filters having an absorption maximum in the UVA (315-400 nm) range, the UVB (280-315 nm) range or the UVC (<280 nm) range that can be used in the cosmetics sector are suitable. UV filters having an absorption maximum in the UVB range, in particular in the range of approximately 280 to approximately 300 nm, are particularly preferred.

The UV filters can, for example, be selected from substituted benzophenones, p-aminobenzoic acid esters, diphenylacrylic acid esters, cinnamic acid esters, salicylic acid esters, benzimidazoles and o-aminobenzoic acid esters.

According to another embodiment of the invention, those UV filters having a cationic group, in particular a quaternary ammonium group, are preferred.

Two preferred UV filters with cationic groups are the compounds cinnamic acid amidopropyltrimethylammonium chloride (Incroquat® UV-283) and dodecyl dimethylaminobenzamidopropyl dimethylammonium tosylate (Escalol® HP 610), which are available as commercial products.

The UV filter or filters (I) are generally used in quantities of 0.1-5 wt. %, based on the overall agent. Quantities of 0.4-2.5 wt. % are preferred.

As well as the essential components and the other above-mentioned preferred components, the cleaning agents according to the invention can in principle contain any other components known to the person skilled in the art for cosmetic agents of this kind.

Other active substances, auxiliary substances and additives are, for example:
other thickeners, such as gelatin or plant gums, for example agar-agar, guar gum, alginates, xanthan gum, gum arabic, karaya gum, locust bean gum, linseed gum, dextrans, starch fractions and derivatives, such as amylose, amylopectin and dextrins, clays and sheet silicates, such as e.g. bentonite, or completely synthetic hydrocolloids, such as e.g. polyvinyl alcohol, the Ca, Mg or Zn soaps,
structurants, such as maleic acid and lactic acid,
solvents and solubility promoters, such as ethanol, isopropanol, ethylene glycol, propylene glycol, glycerol and diethylene glycol,
fiber-structure improving active substances, in particular mono-, di- and oligosaccharides, such as e.g. glucose, galactose, fructose, fruit sugar and lactose,
dyes for coloring the agent,
other substances for adjusting the pH, such as e.g. α- and β-hydroxycarboxylic acids,
active substances, such as allantoin and bisabolol,
complexing agents, such as EDTA, NTA, β-alanine diacetic acid and phosphonic acids,
ceramides. Ceramides are understood to be N-acylsphingosine (fatty acid amides of sphingosine) or synthetic analogs of these lipids (so-called pseudo-ceramides),
opacifiers, such as latex, styrene/PVP and styrene/acrylamide copolymers,
pearlescent agents, such as ethylene glycol mono- and distearate and PEG-3 distearate,
pigments,
propellants, such as propane-butane mixtures, N₂O, dimethyl ether, CO₂ and air,
viscosity regulators, such as salts (NaCl).

The invention secondly provides the use of the cleaning agent according to the invention for the cleaning and care of skin and hair.

The cleaning agents of the present invention foam rapidly in combination with water and provide a large quantity of foam. The foam is stable and easy to distribute on the skin/hair. As a result of its small pore size, the foam feels creamy, allowing the care effect to become palpable during the cleaning operation. Moreover, the foam can be rinsed off rapidly and thoroughly after cleaning, without leaving a slippery feeling on the application surface.

The cleaning agents according to the invention are, furthermore, stable and do not exhibit any separation phenomena even with prolonged storage.

### EXAMPLES

The following cleaning composition according to the invention was produced:

| Raw material | Quantity |
|---|---|
| Fatty alcohol polyglycol ether sulfate | 10 |
| (C12-14), 2EO, 70% AS | |
| Comperlan ®¹ 100 | 0.5 |
| Cocamidopropyl Betaine 40% AS | 10 |
| Polyethylene glycol (PEG-32) | 0.25 |
| White Tea Herbasol Extract ®² | 0.1 |
| Benecel ®³ MP 333C | 0.25 |
| Sodium benzoate | 0.4 |
| Cetiol ®⁴ HE | 0.5 |
| CP Styrene-Acrylic Acid OP 40% | 1 |
| Citric acid | 0.25 |
| NaCl | 0.4 |
| Perfume | 1 |
| Water | to 100 |

After cleaning with the cleaning composition according to the invention, the skin felt soft and nourished.

The cleaning composition foamed very readily and was easy to distribute on the skin.

The cleaning composition was tested by a group of experts for its foam properties, such as quantity of foam, pore size, creaminess, durability, stability and rinsability of the foam, in a blind test in comparison with a market formulation which, instead of the nonionic cellulose ether b) (Benecel®³ MP 333C) and the nonionic polymer c) (PEG-32), contains a cationic polymer.

The blind test showed that the mixture of active substances in the cleaning preparation according to the invention makes the additional incorporation of classic care components, such as cationic polymers, superfluous, as the formulation according to the invention and the cationic polymer-based formulation were perceived by the experts as equivalent with respect to the above-mentioned properties.

The following commercial products were used:
1 INCI name: Cocamide MEA; Cognis
2 INCI name: Isopropylmyristate, Camellia Sinensis Leaf Extract; Cosmetochem,
3 INCI name: Hydroxypropyl Methylcellulose; Hercules
4 INCI name: PEG-7 Glyceryl Cocoate; Cognis

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic cleaning agent, comprising:
a) at least one mild anionic and at least one mild amphoteric/zwitterionic surfactant, wherein the total quantity of mild anionic and mild amphoteric/zwitterionic surfactant(s) is 3 to 20 wt. %,
b) 0.005 to 5 wt. % of at least one nonionic cellulose ether and
c) 0.005 to 5 wt. % of at least one nonionic polymer of formula (I) 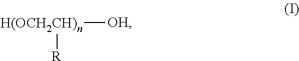
in which R denotes a hydrogen atom or a methyl group and n denotes an average value of 10 to 1200.

2. The cleaning agent according to claim 1, wherein the ratio of anionic surfactant(s) to the amphoteric/zwitterionic surfactant(s) is 3:1 to 1:2, preferably 2.5:1 to 1:1.5 and in particular 2:1 to 1:1.

3. The cleaning agent according to claim 1, wherein the at least one anionic surfactant is at least one surfactant selected from the group consisting of alkyl polyglycol ether sulfates, ether carboxylic acids and sulfosuccinic acid monoalkyl polyoxyethyl esters with 8 to 18 C atoms in the alkyl group and 1 to 10 oxyethyl groups, and the at least one amphoteric/zwitterionic surfactant is at least one surfactant selected from the group consisting of C₈₋₁₈ alkyl betaines, C₈₋₁₈ alkyl amido(C₁₋₄)alkyl betaines and C₈₋₁₈ alkyl amphomonoacetates or amphodiacetates.

4. The cleaning agent according to claim 1, comprising, based on its weight, 0.01 to 4 wt. % nonionic cellulose ether b) selected from the group consisting of hydroxyethyl cellulose, hydroxypropyl cellulose, hydroxypropyl methyl cellulose and mixtures thereof.

5. The cleaning agent according to claim 4, comprising hydroxypropyl methyl cellulose as component b).

6. The cleaning agent according to claim 1, comprising, based on its weight, 0.01 to 4 wt. % nonionic polymer c) of formula (I) selected from the group of the polyethylene glycols in which n has a value of 10 to 1200.

7. The cleaning agent according to claim 1, further comprising, based on its weight, 0.01 to 5 wt. % alkanolamide of formula (II), 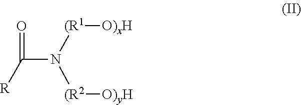
in which R is a C₈-C₂₄ saturated or unsaturated, linear or crosslinked aliphatic group, R₁ and R₂ are the same or different and form a C₂-C₄ linear or branched aliphatic group, x has a value of 0 to 10 and y has a value of 1 to 10, wherein the sum of x+y is less than or equal to 10.

8. The cleaning agent according to claim 1, further comprising at least one solubilizer selected from the group consisting of monoesters and mixtures of monoesters and diesters of glycerol with branched or straight-chained, saturated or unsaturated fatty acids having a C chain length of 8 to 24, which have a degree of ethoxylation of 1 to 20.
